(19) United States
(12) Patent Application Publication
(10) Pub. No.: US 2025/0283029 A1
(43) Pub. Date: Sep. 11, 2025

(54) ENGINEERED MICROORGANISMS WITH ENHANCED PROTEIN EXPRESSION AND SECRETION

(71) Applicant: North Carolina State University, Raleigh, NC (US)

(72) Inventors: Nathan Crook, Raleigh, NC (US); Deniz Durmusoglu, Raleigh, NC (US); Ibrahim Al'Abri, Raleigh, NC (US)

(21) Appl. No.: 18/862,460

(22) PCT Filed: May 1, 2023
(86) PCT No.: PCT/US2023/020551

§ 371 Date: Nov. 1, 2024

Related U.S. Application Data
(60) Provisional application No. 63/337,397, filed on May 2, 2022.

(51) Int. Cl. C12N 1/18; A23L 31/10; C12P 21/00

(52) U.S. Cl. CPC C12N 1/185; A23L 31/10; C12P 21/00

(57) ABSTRACT

The present disclosure provides materials and methods related to the production of peptides and polypeptides from engineered microorganisms. In particular, the present disclosure provides compositions and methods for producing a genetically modified microorganism (e.g., yeast cell) with enhanced protein expression and/or secretion.

### CROSS REFERENCE TO RELATED APPLICATIONS

This application claims priority to and the benefit of U.S. Provisional Patent Application No. 63/337,397 filed May 2, 2022, which is incorporated herein by reference in its entirety for all purposes.

### GOVERNMENT FUNDING

This invention was made with government support under grant number CBET1934284 awarded by the National Science Foundation. The government has certain rights in the invention.

### FIELD

The present disclosure provides materials and methods related to the production of peptides and polypeptides from engineered microorganisms. In particular, the present disclosure provides compositions and methods for producing a genetically modified microorganism (e.g., yeast cell) with enhanced protein expression and/or secretion.

### BACKGROUND

Engineered live biotherapeutics (LBP) are an emerging therapeutic modality where bacterial, fungal, and mammalian cells are engineered to deliver therapeutic payloads to a desired disease location. For example, the majority of currently engineered LBPs for gastrointestinal (GI) diseases have been established in bacterial chassis, such as Lactobacillus spp, Lactococcus spp, and Escherichia coli Nissle 1917. However, yeast probiotics pose a significant biomanufacturing advantage over bacteria, including high protein secretion capacity, sophisticated post-translational protein modification mechanisms, and innate resistance to bacteriophages. However, the use of genetically modified yeast cells as a therapeutic protein production platform has not yet been successfully established, in part because of limitations pertaining to protein secretion capacity. Improving this aspect of protein production technology is important because, for example, after an engineered organism reaches a desired location along the gastrointestinal tract (GIT) and starts establishing there, the physiological challenges present in the GIT may limit its native secretion titers along with other metabolic features, hindering the potency and efficacy of the therapeutic cargo. Therefore, engineered strains with improved secretion arc desirable to ensure production and delivery of therapeutic payloads at desired titers. In addition to utility as an engineered LBP, these strains would also be useful in other areas of biomanufacturing.

### SUMMARY

Embodiments of the present disclosure include an engineered yeast cell comprising at least one genetic modification that reduces activity and/or expression of one or more of the following genes: APE1, YPS1, PRB1, PEP4, PAH1, DER1, HRD1, OCH1, MNN9, VPS5, VPS17, TDA3, GOS1, and ROX1; and at least one exogenous polynucleotide encoding a target polypeptide or protein. In accordance with these embodiments, expression of the target polypeptide or protein is increased compared to a yeast cell lacking the at least one genetic modification.

In some embodiments, the engineered yeast cell is from a probiotic strain. In some embodiments, the yeast cell is selected from the group consisting of S. boulardii, S. cerevisiae, P. pastoris, Y. lipolytica, C. albicans, K. marxianus, Debaryomyces hansenii, and Kluyveromyces lactis. In some embodiments, the yeast cell is from an S. boulardiiΔura3 strain (DD277).

In some embodiments, the at least one genetic modification is a genetic knockout. In some embodiments, the at least one genetic modification comprises a genetic knockout of YPS1. In some embodiments, the at least one genetic modification comprises a genetic knockout of PRB1. In some embodiments, the at least one genetic modification comprises a genetic knockout of PEP4. In some embodiments, the at least one genetic modification comprises a genetic knockout of APE1. In some embodiments, the at least one genetic modification comprises a genetic knockout of PRB1 and PEP4. In some embodiments, the at least one genetic modification comprises a genetic knockout of YPS1, PRB1, PEP4, and APE1.

In some embodiments, the exogenous polynucleotide comprises a promoter upstream of the target polypeptide or protein. In some embodiments, the exogenous polynucleotide comprises a secretion signal upstream of the target polypeptide or protein. In some embodiments, the secretion signal comprises an alpha mating factor secretion signal, an invertase secretion signal, a YAP3-TA57 secretion signal, a preOST1-proαMF (I) secretion signal, or a preOST1-proαMF (MUT1) secretion signal, including any combinations thereof.

In some embodiments, the target polypeptide or protein is expressed and secreted from the engineered yeast cell. In some embodiments, the target polypeptide or protein is expressed on the surface of the engineered yeast cell.

In some embodiments, expression or secretion of the target polypeptide or protein is increased at least 1.2-fold compared to a yeast cell lacking the at least one genetic modification.

In some embodiments, the target polypeptide or protein is at least one of an anti-cancer agent, an immune regulator, an anti-pathogenic agent, and/or a metabolic regulator.

In some embodiments, the target polypeptide or protein is at least one of a DARPin, a lectin, a monoclonal antibody, a F(ab′)₂ fragment, a Fab′/Fab fragment, a diabody, a scFv, a nanobody, and/or an affibody.

Embodiments of the present disclosure also include a composition comprising any of the engineered yeast cells described herein.

In some embodiments, the composition is lyophilized. In some embodiments, the composition is in wet form. In some embodiments, the composition is in frozen form.

In some embodiments, the composition is formulated as a food product.

Embodiments of the present disclosure also include a method of treating and/or preventing a disease or condition in a subject. In accordance with these embodiments, the method includes administering any of the compositions described herein to the subject.

In some embodiments, the engineered yeast cells are present in the composition at a dose from about 1×10⁵ cells/kg body weight to about 1×10¹⁵ cells/kg body weight of the subject.

In some embodiments, the composition is administered orally or rectally.

Embodiments of the present disclosure also include a method of enhancing expression and/or secretion of a target polypeptide or protein in an engineered yeast cell. In accordance with these embodiments, the method includes making at least one genetic modification to the engineered yeast cell that reduces activity and/or expression of one or more of the following genes: APE1, YPS1, PRB1, PEP4, PAH1, DER1, HRD1, OCH1, MNN9, VPS5, VPS17, TDA3, GOS1, and ROX1. In some embodiments, expression of the target polypeptide or protein is increased compared to a yeast cell lacking at least one genetic modification.

### BRIEF DESCRIPTION OF THE DRAWINGS

FIGS. 1A-1B: Representative schematic for generating engineered yeast cells with exogenous polynucleotide constructs and corresponding protein expression/secretion for testing various genetic modifications (FIG. 1A); a representative time course for detecting protein expression from the exogenous polynucleotide constructs in the engineered yeast cells (FIG. 1B).

FIG. 2: Representative graphical data demonstrating the generation and testing of low copy (CEN6/ARS4) and genomic integration (chromosomal) polynucleotide constructs in the engineered yeast cells of the present disclosure (levels of protein expression/secretion are independent of cell concentration).

FIG. 3: Representative graphical data demonstrating the generation and testing of exogenous polynucleotide constructs and corresponding protein expression/secretion using various secretion signals in the engineered yeast cells of the present disclosure, including alpha mating factor secretion signal (αMFprepro), alpha mating factor signal pre-and invertase secretion signal (SUC2), YAP3-TA57 secretion signal, preOST1-proαMF (I) secretion signal, and preOST1-pro α MF (MUT1) secretion signal (levels of protein expression/secretion are independent of cell concentration).

FIG. 4: Representative graphical data of the generation and testing of the various engineered yeast cells of the present disclosure containing a genetic modification in each of the following genes: APE1, YPS1, PRB1, PEP4, PAH1, DER1, HRD1, OCH1, MNN9, VPS5, VPS17, TDA3, GOS1, or ROX1 (levels of protein expression/secretion are independent of cell concentration).

FIG. 5: Representative graphical data of the generation and testing of the various engineered yeast cells of the present disclosure containing a genetic modification in one or more of the following genes: APE1, YPS1, PRB1, PEP4, PAH1, DER1, HRD1, OCH1, MNN9, VPS5, VPS17, TDA3, GOS1, or ROX1 (levels of protein expression/secretion are independent of cell concentration).

FIG. 6: Representative graphical data of the generation and testing of the engineered yeast cells of the present disclosure containing genetic modifications in APE1, YPS1, PRB1, and PEP4, and either genomic integration or high copy exogenous polynucleotide constructs (levels of protein expression/secretion are independent of cell concentration).

### DETAILED DESCRIPTION

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The use of the terms “a” and “an” and “the” and “at least one” and similar referents in the context of describing the embodiments of the present disclosure (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the various embodiments of the present disclosure, and does not pose a limitation on the scope of these embodiment unless otherwise claimed. No language in the specification should be construed as indicating any non-claimed element as essential to the practice of the various embodiments of the present disclosure.

As used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

The transitional phrase “consisting essentially of” as used in claims in the present application limits the scope of a claim to the specified materials or steps “and those that do not materially affect the basic and novel characteristic(s)” of the claimed invention, as discussed in In re Herz, 537 F.2d 549, 551-52, 190 USPQ 461, 463 (CCPA 1976). For example, a composition “consisting essentially of” recited elements may contain an unrecited contaminant at a level such that, though present, the contaminant does not alter the function of the recited composition as compared to a pure composition, i.e., a composition “consisting of” the recited components.

The term “one or more,” as used herein, refers to a number higher than one. For example, the term “one or more” encompasses any of the following: two or more, three or more, four or more, five or more, six or more, seven or more, eight or more, nine or more, ten or more, eleven or more, twelve or more, thirteen or more, fourteen or more, fifteen or more, twenty or more, fifty or more, 100 or more, or an even greater number.

The term “one or more but less than a higher number,” “two or more but less than a higher number,” “three or more but less than a higher number,” “four or more but less than a higher number,” “five or more but less than a higher number,” “six or more but less than a higher number,” “seven or more but less than a higher number,” “eight or more but less than a higher number,” “nine or more but less than a higher number,” “ten or more but less than a higher number,” “eleven or more but less than a higher number,” “twelve or more but less than a higher number,” “thirteen or more but less than a higher number,” “fourteen or more but less than a higher number,” or “fifteen or more but less than a higher number” is not limited to a higher number. For example, the higher number can be 10,000, 1,000, 100, 50, etc. For example, the higher number can be approximately 50 (e.g., 50, 49, 48, 47, 46, 45, 44, 43, 42, 41, 40, 39, 38, 37, 36, 35, 34, 33, 32, 31, 32, 30, 29, 28, 27, 26, 25, 24, 23, 22, 21, 20, 19, 18, 17, 16, 15, 14, 13, 12, 11, 10, 9, 8, 7, 6, 5, 4, 3 or 2).

“Correlated to” as used herein refers to compared to.

As The terms “nucleic acid,” “polynucleotide,” “nucleotide sequence,” and “oligonucleotide” are used interchangeably herein and refer to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The terms encompass any deoxyribonucleotide, ribonucleotide, or peptide nucleic acid component, and any chemical variants thereof, such as methylated, hydroxymethylated, or glycosylated forms of these bases. The polymers or oligomers may be heterogenous or homogenous in composition, may be isolated from naturally occurring sources, or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002) and U.S. Pat. No. 5,034,506), locked nucleic acid (LNA; see Wahlestedt et al., Proc. Natl. Acad. Sci. U.S.A., 97:5633-5638 (2000)), cyclohexenyl nucleic acids (see Wang, J. Am. Chem. Soc., 122:8595-8602 (2000)), and/or a ribozyme. The terms “nucleic acid” and “nucleic acid sequence” may also encompass a chain comprising non-natural nucleotides, modified nucleotides, and/or non-nucleotide building blocks that can exhibit the same function as natural nucleotides (e.g., “nucleotide analogs”).

The terms “peptide,” “polypeptide,” and “protein” are used interchangeably herein and refer to a polymeric form of amino acids of any length, which can include coded and non-coded amino acids, chemically or biochemically modified or derivatized amino acids, and polypeptides having modified peptide backbones.

As used herein, a “nucleic acid” or “nucleic acid molecule” generally refers to any ribonucleic acid or deoxyribonucleic acid, which may be unmodified or modified DNA or RNA. “Nucleic acids” include, without limitation, single-and double-stranded nucleic acids. As used herein, the term “nucleic acid” also includes DNA as described above that contains one or more modified bases. Thus, DNA with a backbone modified for stability or for other reasons is a “nucleic acid.” The term “nucleic acid” as it is used herein embraces such chemically, enzymatically, or metabolically modified forms of nucleic acids, as well as the chemical forms of DNA characteristic of viruses and cells, including for example, simple and complex cells.

The terms “oligonucleotide” or “polynucleotide” or “nucleotide” or “nucleic acid” refer to a molecule having two or more deoxyribonucleotides or ribonucleotides, preferably more than three, and usually more than ten. The exact size will depend on many factors, which in turn depends on the ultimate function or use of the oligonucleotide. The oligonucleotide may be generated in any manner, including chemical synthesis, DNA replication, reverse transcription, or a combination thereof. Typical deoxyribonucleotides for DNA are thymine, adenine, cytosine, and guanine. Typical ribonucleotides for RNA are uracil, adenine, cytosine, and guanine.

The terms “complementary” and “complementarity” refer to nucleotides (e.g., 1 nucleotide) or polynucleotides (e.g., a sequence of nucleotides) related by the base-pairing rules. For example, the sequence 5′-A-G-T-3′ is complementary to the sequence 3′-T-C-A-5′. Complementarity may be “partial,” in which only some of the nucleic acids' bases are matched according to the base pairing rules. Or, there may be “complete” or “total” complementarity between the nucleic acids. The degree of complementarity between nucleic acid strands affects the efficiency and strength of hybridization between nucleic acid strands. This is of particular importance in amplification reactions and in detection methods that depend upon binding between nucleic acids.

The term “gene” refers to a nucleic acid (e.g., DNA or RNA) sequence that comprises coding sequences necessary for the production of an RNA, or of a polypeptide or its precursor. A functional polypeptide can be encoded by a full-length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a “gene” may comprise fragments of the gene or the entire gene.

The term “gene” also encompasses the coding regions of a structural gene and includes sequences located adjacent to the coding region on both the 5′ and 3′ ends, e.g., for a distance of about 1 kb on either end, such that the gene corresponds to the length of the full-length mRNA (e.g., comprising coding, regulatory, structural and other sequences). The sequences that are located 5′ of the coding region and that are present on the mRNA are referred to as 5′ non-translated or untranslated sequences. The sequences that are located 3′ or downstream of the coding region and that are present on the mRNA are referred to as 3′ non-translated or 3′ untranslated sequences. The term “gene” encompasses both cDNA and genomic forms of a gene. In some organisms (e.g., eukaryotes), a genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ ends of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, posttranscriptional cleavage, and polyadenylation.

The term “wild-type” when made in reference to a gene refers to a gene that has the characteristics of a gene isolated from a naturally occurring source. The term “wild-type” when made in reference to a gene product refers to a gene product that has the characteristics of a gene product isolated from a naturally occurring source. The term “wild-type” when made in reference to a protein refers to a protein that has the characteristics of a naturally occurring protein. The term “naturally-occurring” as applied to an object refers to the fact that an object can be found in nature. For example, a polypeptide or polynucleotide sequence that is present in an organism (including viruses) that can be isolated from a source in nature, and which has not been intentionally modified by the hand of a person in the laboratory is naturally-occurring. A wild-type gene is often that gene or allele that is most frequently observed in a population and is thus arbitrarily designated the “normal” or “wild-type” form of the gene. In contrast, the term “modified” or “mutant” when made in reference to a gene or to a gene product refers, respectively, to a gene or to a gene product that displays modifications in sequence and/or functional properties (e.g., altered characteristics) when compared to the wild-type gene or gene product. It is noted that naturally-occurring mutants can be isolated; these are identified by the fact that they have altered characteristics when compared to the wild-type gene or gene product.

The term “allele” refers to a variation of a gene; the variations include but are not limited to variants and mutants, polymorphic loci, and single nucleotide polymorphic loci, frameshift, and splice mutations. An allele may occur naturally in a population, or it might arise during the lifetime of any particular individual of the population.

Thus, the terms “variant” and “mutant” when used in reference to a nucleotide sequence refer to a nucleic acid sequence that differs by one or more nucleotides from another, usually related, nucleotide acid sequence. A “variation” is a difference between two different nucleotide sequences; typically, one sequence is a reference sequence.

### 2. COMPOSITIONS AND METHODS

Embodiments of the present disclosure include an engineered yeast cell comprising at least one genetic modification that enhances protein expression and/or secretion of a target protein or polypeptide. In accordance with these embodiments, the present disclosure provides engineered yeast cells that include one or more genetic modifications in one or more genes that are involved in protein expression and/or secretion. These engineered yeast cells can be used as a platform to, for example, generate engineered live biotherapeutics (LBPs) to be administered to a subject to treat a disease or condition, and/or as a biomanufacturing platform to generate biologics for the treatment of a disease or condition. As would be recognized by one of ordinary skill in the art, the genetically modified yeast strains of the present disclosure can be used to express/secrete any desired protein or polypeptide. In some embodiments, the engineered yeast cell is from a probiotic strain. In some embodiments, the yeast cell is selected from the group consisting of S. boulardii, S. cerevisiae, P. pastoris, Y. lipolytica, C. albicans, K. marxianus, Debaryomyces hansenii, and Kluyveromyces lactis. In some embodiments, the yeast cell is from an S. boulardiiΔura3 strain (DD277).

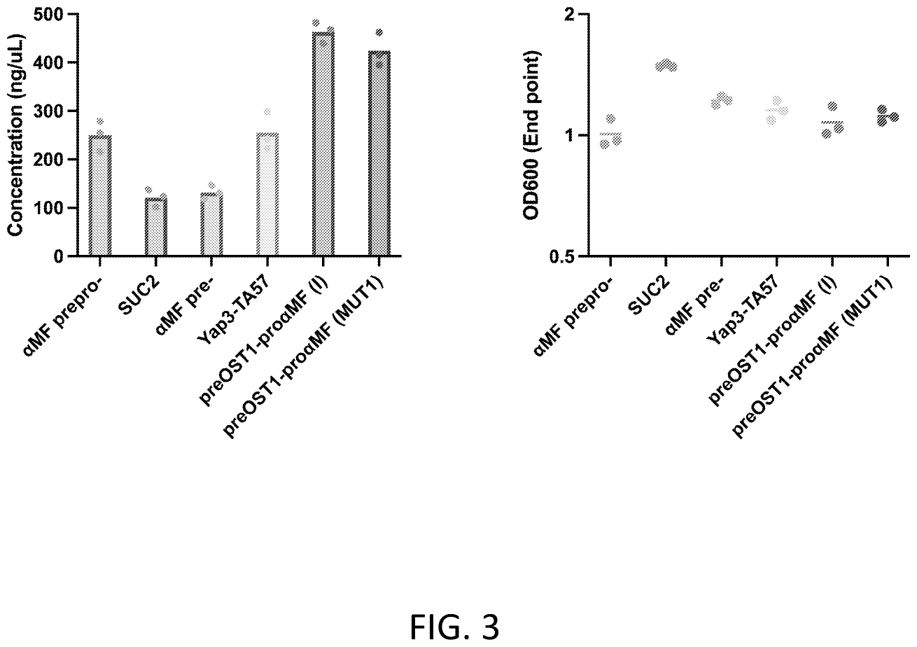

In some embodiments, the engineered yeast cells of the present disclosure include at least one genetic modification in a gene involved in the protein secretion pathway. In some embodiments, the genetic medication results in the reduction of the activity and/or expression of the gene(s) involved in the protein secretion pathway. In some embodiments, the genetic modification is a gene knockout or a loss-of-function mutation. As described further herein, the genetic modification can be in any one of the following genes: APE1, YPS1, PRB1, PEP4, PAH1, DER1, HRD1, OCH1, MNN9, VPS5, VPS17, TDA3, GOS1, and ROX1. In accordance with these embodiments, expression of a target polypeptide or protein is increased compared to a yeast cell lacking at least one genetic modification in one or more of these genes (FIG. 3).

As would be recognized by one of ordinary skill in the art based on the present disclosure, the gene APE1 encodes Vacuolar aminopeptidase 1 (Uniprot No. P14904). The YPS1 gene encodes Aspartic proteinase 3 (Uniprot No. P32329). The PRB1 gene encodes Cerevisin (Uniprot No. P09232). The PEP4 gene encodes Vacuolar proteinase A (Uniprot No. A6ZW99). The PAH1 gene encodes Phosphatidic acid phosphohydrolase 1 (Uniprot No. P32567). The DER1 gene encodes Degradation in the endoplasmic reticulum protein 1 (Uniprot No. P38307). The HRD1 gene encodes ERAD-associated E3 ubiquitin-protein ligase (Uniprot No. Q08109). The OCH1 gene encodes Initiation-specific alpha-1,6-mannosyltransferase (Uniprot No. P31755). The MNN9 gene encodes Mannan polymerase complexes subunit MNN9 (Uniprot No. P39107). The VPS5 gene encodes Vacuolar protein sorting-associated protein vps5 (Uniprot No. Q9COU7). The VPS17 gene encodes Vacuolar protein sorting-associated protein 17 (Uniprot No. A6ZNX9). The TDA3 gene encodes Putative oxidoreductase TDA3 (Uniprot No. P38758). The GOS1 gene encodes Golgi SNAP receptor complex member 1 (Uniprot No. P38736). The ROX1 gene encodes Repressor ROX1 (Uniprot No. P25042).

A variety of genetic modifications (e.g., loss-of-function mutations) can be used in the various compositions and methods described herein. A genetic locus in a microorganism can produce one or more gene products. In some embodiments, the gene product is a nucleic acid, for example a ribosomal RNA. In some embodiments, the gene product is a polypeptide. A genetic modification affecting one or more genetic loci can cause a loss-of-function in the gene product, a gain-of-function in the gene product, and/or can cause the gene product to adopt a new function. In some embodiments, a loss-of-function mutation is provided that reduces, but does not eliminate activity of the gene product (this class of mutation can also be referred to herein a “hypomorphic” mutation or “partial loss-of-function” mutation). In some embodiments, a loss-of-function mutation substantially reduces gene product activity. As used herein, “substantially reduces” gene product activity and variations of this root term refer to at least a 50% reduction in gene product activity compared to wild-type, for example, a 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%, 99.99%, or 99.999% reduction. In some embodiments, a loss-of-function mutation eliminates gene product activity (this class of mutation can also be referred to herein as a “null” mutation). In some embodiments, a null mutation eliminates at least the gene-product-encoding sequence of a genetic locus. In some embodiments a null mutation eliminates the entire genetic locus. While a deletion of a genetic locus is a type of null mutation, null mutations can also encompass other sorts of genetic modifications. In some embodiments, a null mutation does not eliminate gene-product-encoding sequence, but prevents expression of the gene product (for example, by eliminating a promoter, a translation start sequence, or introducing an early stop codon). In some embodiments, a null mutation does not climinate gene-product-encoding sequence or expression of a gene product, but eliminates or substantially eliminates activity of the gene product (for example, by mutating one or more catalytic residues from a protein). This latter class of null mutation may also be referred to herein as a “phenotypic null,” or variations of this root term. In some embodiments, a loss-of-function mutation is a dominant negative mutation.

In addition to mutation of a genetic locus, other genetic modifications can also reduce or eliminate gene activity. By way of non-limiting example, antisense oligonucleotides can reduce or eliminate activity of the target gene. In some embodiments, a genetic modification for reducing gene expression comprises at least one antisense oligonucleotide. In some embodiments, an antisense oligonucleotide comprises an RNA complementary to at least a portion of a transcript of a target gene. In some embodiments, a yeast cell is genetically modified to express an antisense RNA directed to at least one transcript of the target gene. Additional exemplary genetic modifications that can be used to reduce gene activity in accordance with some embodiments herein include ribozymes, transcriptional repressors, inducible promoters, proteases directed to polypeptide encoded by a target gene, and the like.

In some embodiments, a mutation or genetic modification eliminates the activity of two or more gene products. In some embodiments, a mutation deletes an operon. In some embodiments, a mutation eliminates activity of one gene, and as a result, also eliminates activity of a second gene (for example, if the products of gene A and gene B function as a dimer, the elimination of either of gene A activity or gene B activity can also eliminate activity of the other gene).

A variety of techniques for making mutations are known to the skilled artisan. In some embodiments, a desired mutation is introduced via homologous recombination. A variety of vectors can be used for homologous mutation, for example phage or viral vectors, plasmid vectors, artificial chromosomes, and the like. In some embodiments, homologous sequences on a vector flank a genetic locus that can be used to identify homologous recombinants, for example an antibiotic resistance marker (for example, but not limited to kanamycin, chloramphenicol, or ampicillin resistance) or metabolic enzyme that permits an auxotroph to survive in a particular minimal medium. In some embodiments, mutations are introduced into a genome randomly, and mutant microorganisms having the desired mutations are selected.

In some embodiments, a host genome or portion thereof is synthesized, and introduced into a microorganism. In some embodiments an entire host genome having the desired genetic features is synthesized and inserted into a yeast cell.

In some embodiments, the at least one genetic modification comprises a genetic knockout of or a loss-of-function mutation in APE1. In some embodiments, the engineered yeast cells comprises a genetic knockout of or a loss-of-function mutation in APE1 and at least one other gene selected from the group consisting of YPS1, PRB1, PEP4, PAH1, DER1, HRD1, OCH1, MNN9, VPS5, VPS17, TDA3, GOS1, and ROX1. In some embodiments, the engineered yeast cell comprises a genetic knockout of or a loss-of-function mutation in APE1 and at least two other genes, at least three other genes, at least four other genes, or at least five other genes selected from the group consisting of YPS1, PRB1, PEP4, PAH1, DER1, HRD1, OCH1, MNN9, VPS5, VPS17, TDA3, GOS1, and ROX1.

In some embodiments, the at least one genetic modification comprises a genetic knockout of or a loss-of-function mutation in APE1. In some embodiments, the engineered yeast cells comprises a genetic knockout of or a loss-of-function mutation in YPS1 and at least one other gene selected from the group consisting of APE1, PRB1, PEP4, PAH1, DER1, HRD1, OCH1, MNN9, VPS5, VPS17, TDA3, GOS1, and ROX1. In some embodiments, the engineered yeast cell comprises a genetic knockout of or a loss-of-function mutation in YPS1 and at least two other genes, at least three other genes, at least four other genes, or at least five other genes selected from the group consisting of APE1, PRB1, PEP4, PAH1, DER1, HRD1, OCH1, MNN9, VPS5, VPS17, TDA3, GOS1, and ROX1.

In some embodiments, the at least one genetic modification comprises a genetic knockout of or a loss-of-function mutation in PRB1. In some embodiments, the engineered yeast cells comprises a genetic knockout of or a loss-of-function mutation in YPS1 and at least one other gene selected from the group consisting of APE1, YPS1, PEP4, PAH1, DER1, HRD1, OCH1, MNN9, VPS5, VPS17, TDA3, GOS1, and ROX1. In some embodiments, the engineered yeast cell comprises a genetic knockout of or a loss-of-function mutation in PRB1 and at least two other genes, at least three other genes, at least four other genes, or at least five other genes selected from the group consisting of APE1, YPS1, PEP4, PAH1, DER1, HRD1, OCH1, MNN9, VPS5, VPS17, TDA3, GOS1, and ROX1.

In some embodiments, the at least one genetic modification comprises a genetic knockout of or a loss-of-function mutation in PEP4. In some embodiments, the engineered yeast cells comprises a genetic knockout of or a loss-of-function mutation in YPS1 and at least one other gene selected from the group consisting of APE1, YPS1, PRB1, PAH1, DER1, HRD1, OCH1, MNN9, VPS5, VPS17, TDA3, GOS1, and ROX1. In some embodiments, the engineered yeast cell comprises a genetic knockout of or a loss-of-function mutation in PEP4 and at least two other genes, at least three other genes, at least four other genes, or at least five other genes selected from the group consisting of APE1, YPS1, PRB1, PAH1, DER1, HRD1, OCH1, MNN9, VPS5, VPS17, TDA3, GOS1, and ROX1.

In some embodiments, the engineered yeast cell of the present disclosure comprises at least one genetic modification comprising a genetic knockout of or a loss-of-function mutation in PRB1 and PEP4. In some embodiments, the engineered yeast cell comprises a genetic knockout of or a loss-of-function mutation in PRB1 and PEP4, and at least one other gene, at least two other genes, at least three other genes, at least four other genes, or at least five other genes selected from the group consisting of APE1, YPS1, PAH1, DER1, HRD1, OCH1, MNN9, VPS5, VPS17, TDA3, GOS1, and ROX1.

In some embodiments, the engineered yeast cell of the present disclosure comprises at least one genetic modification comprising a genetic knockout of or a loss-of-function mutation in YPS1, PRB1, PEP4, and APE1. In some embodiments, the engineered yeast cell comprises a genetic knockout of or a loss-of-function mutation in YPS1, PRB1, PEP4, and APE1, and at least one other gene, at least two other genes, at least three other genes, at least four other genes, or at least five other genes selected from the group consisting of PAH1, DER1, HRD1, OCH1, MNN9, VPS5, VPS17, TDA3, GOS1, and ROX1.

In accordance with these embodiments, the present disclosure provides an engineered yeast cell comprising one of the genetic modifications described above, as well as at least one exogenous polynucleotide that encodes a target polypeptide or protein. In some embodiments, at least a portion of the exogenous polynucleotide encodes a protein or polypeptide that is endogenous to the engineered yeast cell. In other embodiments, at least a portion of the exogenous polynucleotide encodes a protein or polypeptide that is exogenous to the engineered yeast cell. In some embodiments, the exogenous polynucleotide encodes more than one protein or polypeptide that can be endogenous and/or exogenous to the engineered yeast cell.

In some embodiments, the exogenous polynucleotide comprises other features that facilitate protein expression and/or secretion in the engineered yeast cell. For example, the exogenous polypeptide can include a promoter upstream of the target polypeptide or protein in order to facilitate its expression. In some embodiments, the exogenous polynucleotide can include a secretion signal upstream of the target polypeptide or protein in order to facilitate its secretion from the engineered yeast cell. Although any suitable promoter or secretion signal can be used, as would be recognized by one of ordinary skill in the art, in some embodiments, the secretion signal comprises an alpha mating factor secretion signal. In some embodiments, the target polypeptide or protein is expressed and secreted from the engineered yeast cell. In some embodiments, the target polypeptide or protein is expressed on the surface of the engineered yeast cell.

In some embodiments, the engineered yeast cells of the present disclosure comprising at least one of the genetic modifications described herein exhibit increased expression, secretion, and/or cell surface display of the target polypeptide or protein. In some embodiments, expression, secretion, and/or cell surface display of the target polypeptide or protein is increased at least 1.1-fold compared to a yeast cell lacking the genetic modification. In some embodiments, expression, secretion, and/or cell surface display of the target polypeptide or protein is increased at least 1.2-fold compared to a yeast cell lacking the genetic modification. In some embodiments, expression, secretion, and/or cell surface display of the target polypeptide or protein is increased at least 1.3-fold compared to a yeast cell lacking the genetic modification. In some embodiments, expression, secretion, and/or cell surface display of the target polypeptide or protein is increased at least 1.4-fold compared to a yeast cell lacking the genetic modification. In some embodiments, expression, secretion, and/or cell surface display of the target polypeptide or protein is increased at least 1.5-fold compared to a yeast cell lacking the genetic modification. In some embodiments, expression, secretion, and/or cell surface display of the target polypeptide or protein is increased at least 1.6-fold compared to a yeast cell lacking the genetic modification. In some embodiments, expression, secretion, and/or cell surface display of the target polypeptide or protein is increased at least 1.7-fold compared to a yeast cell lacking the genetic modification. In some embodiments, expression, secretion, and/or cell surface display of the target polypeptide or protein is increased at least 1.8-fold compared to a yeast cell lacking the genetic modification. In some embodiments, expression, secretion, and/or cell surface display of the target polypeptide or protein is increased at least 1.9-fold compared to a yeast cell lacking the genetic modification. In some embodiments, expression, secretion, and/or cell surface display of the target polypeptide or protein is increased at least 2.0-fold compared to a yeast cell lacking the genetic modification. In some embodiments, expression, secretion, and/or cell surface display of the target polypeptide or protein is increased at least 3.0-fold compared to a yeast cell lacking the genetic modification. In some embodiments, expression, secretion, and/or cell surface display of the target polypeptide or protein is increased at least 4.0-fold compared to a yeast cell lacking the genetic modification. In some embodiments, expression, secretion, and/or cell surface display of the target polypeptide or protein is increased at least 5.0-fold compared to a yeast cell lacking the genetic modification. In some embodiments, expression, secretion, and/or cell surface display of the target polypeptide or protein is increased at least 6.0-fold compared to a yeast cell lacking the genetic modification. In some embodiments, expression, secretion, and/or cell surface display of the target polypeptide or protein is increased at least 7.0-fold compared to a yeast cell lacking the genetic modification. In some embodiments, expression, secretion, and/or cell surface display of the target polypeptide or protein is increased at least 8.0-fold compared to a yeast cell lacking the genetic modification. In some embodiments, expression, secretion, and/or cell surface display of the target polypeptide or protein is increased at least 9.0-fold compared to a yeast cell lacking the genetic modification. In some embodiments, expression, secretion, and/or cell surface display of the target polypeptide or protein is increased at least 10.0-fold compared to a yeast cell lacking the genetic modification. In some embodiments, expression, secretion, and/or cell surface display of the target polypeptide or protein is increased at least 15.0-fold compared to a yeast cell lacking the genetic modification. In some embodiments, expression, secretion, and/or cell surface display of the target polypeptide or protein is increased at least 20.0-fold compared to a yeast cell lacking the genetic modification.

Although any target protein or polypeptide can be expressed in the engineered yeast cells of the present disclosure, in some embodiments, the target polypeptide or protein is an anti-cancer agent, an immune regulator, an anti-pathogenic agent, and/or a metabolic regulator. In some embodiments, the target polypeptide or protein is at least one of a DARPin, a lectin, a monoclonal antibody, a F(ab′)₂ fragment, a Fab′/Fab fragment, a diabody, a scFv, a nanobody, and/or an affibody.

The term “target protein” as used herein refers to a polypeptide or protein produced by recombinant technology in a host cell. More specifically, the protein may be a polypeptide that does not occur naturally in the host cell (i.e., a heterologous protein), or may be natural to the host cell (i.e., a protein homologous to the host cell), for example, by transformation with a self-replicating vector containing a nucleic acid sequence encoding a target protein, or by integration of one or more copies of the nucleic acid sequence encoding a target protein into the genome of a host cell, or by integration techniques, e.g., a promoter produced by recombinant modification of one or more regulatory sequences that regulate expression of the gene encoding the target protein. In some embodiments, the target protein is preferably an antibody or fragment thereof, enzyme and peptide, protein antibiotic, toxin fusion protein, carbohydrate-protein conjugate, structural protein, regulatory protein, vaccine and vaccine-like protein or particle. The target protein can also be a recombinant or heterologous protein selected from therapeutic proteins including process enzymes, growth factors, hormones and cytokines or metabolites of target protein. An exemplary target protein is an antigen-binding molecule such as an antibody or fragment thereof. Among certain target proteins are monoclonal antibodies (mAb), immunoglobulins (Ig) or immunoglobulin class G (IgG), heavy chain antibodies (HcAb′), or fragments thereof, such as fragment-antigen binding (Fab), Fd, Single-chain variable fragments (scFv) or engineered variants thereof, such as Fv dimer (diabody), Fv trimer (tribody), Fv tetramer or minibody, and VH or VHH or V-NAR Antibodies such as single-domain antibodies.

In accordance with the above embodiments, the present disclosure also includes a composition comprising any of the engineered yeast cells described herein. In some embodiments, the composition is formulated as a food product or a medicament. In some embodiments, the composition is lyophilized. In some embodiments, the composition is in wet form. In some embodiments, the composition is in frozen form. As would be recognized by one of ordinary skill in the art based on the present disclosure, yeast cells can be formulated as a lyophilized composition (e.g., including a cryoprotectant) and can be readily reconstituted, which is an advantage over many other microorganisms (e.g., bacteria and fungi). In this aspect, the compositions of the present disclosure are particularly suited for lyophilization and formulation as an LBP and/or therapeutic composition for administration to a subject.

Generally, the nomenclature used herein and the laboratory procedures utilized in the present disclosure include molecular, biochemical, microbiological and recombinant DNA techniques that are well known and commonly employed in the art. Standard techniques are used for cloning, DNA and RNA isolation, amplification and purification. Generally enzymatic reactions involving DNA ligase, DNA polymerase, restriction endonucleases and the like are performed according to the manufacturer's specifications. Such techniques are thoroughly explained in the literature and are generally performed according to methods available to those of skill in the art. For example, the phrase “nucleic acid” or “polynucleotide sequence” refers to a single-stranded or double-stranded polymer of deoxyribonucleotide or ribonucleotide bases read from the 5′ to the 3′ end. Nucleic acids may also include modified nucleotides that permit correct read-through by a polymerase and do not alter expression of a polypeptide encoded by that nucleic acid. A “coding sequence” or “coding region” refers to a nucleic acid molecule having sequence information necessary to produce a gene product, when the sequence is expressed. The phrase “nucleic acid sequence encoding” refers to a nucleic acid which directs the expression of a specific protein or polypeptide. The nucleic acid sequences described in the present disclosure include both the DNA strand sequence that is transcribed into RNA and the RNA sequence that is translated into protein. The nucleic acid sequences include both the full length nucleic acid sequences as well as non-full length sequences derived from the full length sequences. It should be understood that the sequences include the degenerate codons of the native sequence or sequences which may be introduced to provide codon preference in a specific host cell.

The terms “isolated,” “purified,” or “biologically pure” refer to material that is substantially or essentially free from components that normally accompany it as found in its native state. Purity and homogeneity are typically determined using molecular biology and analytical chemistry techniques such as polyacrylamide gel electrophoresis or high performance liquid chromatography. A protein that is the predominant species present in a preparation is substantially purified. In particular, an isolated nucleic acid of the present disclosure is separated from open reading frames that flank the desired gene and encode proteins other than the desired protein. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

An “expression cassette” refers to a nucleic acid construct, which when introduced into a host cell, results in transcription and/or translation of an RNA and/or polypeptide, respectively. The expression cassette may include a nucleic acid comprising a promoter sequence, with or without a sequence containing mRNA polyadenylation signals, and one or more restriction enzyme sites located downstream from the promoter allowing insertion of heterologous gene sequences. The expression cassette is capable of directing the expression of a heterologous protein when the gene encoding the heterologous protein is operably linked to the promoter by insertion into one of the restriction sites. The recombinant expression cassette allows expression of the heterologous protein in a host cell when the expression cassette containing the heterologous protein is introduced into the host cell. Expression cassettes can be derived from a variety of sources depending on the host cell to be used for expression. For example, an expression cassette can contain components derived from a viral, bacterial, insect, plant, or mammalian source. In the case of both expression of transgenes and inhibition of endogenous genes (e.g., by antisense, or sense suppression) the inserted polynucleotide sequence need not be identical and can be “substantially identical” to a sequence of the gene from which it was derived. For example, some yeasts belong to the CUG clade which have an alternative codon usage and the polynucleotide must be altered to code for the correct amino acid and ensure that lys/ser are properly incorporated.

The term “recombinant cell” (or simply “host cell”) refers to a cell into which a recombinant expression vector containing the constructs described herein has been introduced. It should be understood that the term “host cell” is intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. Methods for introducing polynucleotide sequences into various types of cells are well known in the art. Provided are host cells or progeny of host cells transformed with the recombinant expression cassettes and constructs of the present disclosure.

The terms “promoter,” “promoter region,” or “promoter sequence” refer generally to transcriptional regulatory regions of a gene, which may be found at the 5′ or 3′ l side of the coding region, or within the coding region, or within introns. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

As used herein, a polynucleotide is “operably linked,” “operably connected,” or “operably inserted” when it is placed into a functional relationship with a second polynucleotide sequence. For instance, a promoter is operably linked to a coding sequence if the promoter is connected to the coding sequence such that it may affect transcription of the coding sequence. This same definition is sometimes applied to the arrangement of other transcription control elements (e.g., enhancers, terminators) in an expression cassette. Transcriptional and translational control sequences are DNA regulatory sequences, such as promoters, enhancers, polyadenylation signals, terminators, and the like, that provide for the expression of a coding sequence in a host cell. Promoters useful in the practice of the present invention include, but are not limited to, constitutive, inducible, temporally-regulated, developmentally regulated, and chemically regulated promoters.

The term “nucleic acid construct” or “DNA construct” is sometimes used to refer to a coding sequence or sequences operably linked to appropriate regulatory sequences and inserted into an expression cassette for transforming a cell or for translating a protein in a cell-free system or for use in homologous recombination. Such a nucleic acid construct may contain a coding sequence for a gene product of interest, and optionally a selectable marker gene and/or a reporter gene. The term “selectable marker gene” refers to a gene encoding a product that, when expressed, confers a selectable phenotype, such as antibiotic resistance, on a transformed cell. The term “reporter gene” refers to a gene that encodes a product which is easily detectable by standard methods, either directly or indirectly. Reporter genes include, but are not limited to luciferases, ß-glucuronidase (GUS), fluorescent proteins such as green fluorescent protein (GFP), dsRed, mCherry and others available to those skilled in the art. Selectable markers include but are not limited to markers that confer resistance to an antibiotic such as kanamycin and hygromycin.

A “heterologous” region of a nucleic acid construct is an identifiable segment (or segments) of the nucleic acid molecule within a larger molecule that is not found in association with the larger molecule in nature. When the heterologous region encodes a gene, the gene will usually be flanked by DNA that does not flank the genetic DNA in the genome of the source organism. In another example, a heterologous region is a construct where the coding sequence itself is not found in nature. The term “DNA construct” is also used to refer to a heterologous region, particularly one constructed for use in transformation of a cell.

The term “vector” is intended to refer to a nucleic acid molecule capable of transporting another nucleic acid to which it has been linked. One type of vector is a “plasmid,” which refers to a circular double-stranded DNA loop into which additional DNA segments may be ligated. Another type of vector is a viral vector, where additional DNA segments may be ligated into the viral genome. Certain vectors are capable of autonomous replication in a host cell into which they are introduced (e.g., bacterial vectors having a bacterial origin of replication and episomal mammalian vectors). Other vectors can be integrated into the genome of a host cell upon introduction into the host cell, and thereby are replicated along with the host genome, such as some viral vectors or transposons. Moreover, certain vectors are capable of directing the expression of genes to which they are operatively linked or connected. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

A “nucleic acid probe” or “oligonucleotide” is defined as a nucleic acid capable of binding to a target nucleic acid of complementary sequence through one or more types of chemical bonds, usually through complementary base pairing, usually through hydrogen bond formation. As used herein, a probe may include natural bases (i.e., A, G, C, or T) or modified bases (7-deazaguanosine, inosine, etc.). In addition, the bases in a probe may be joined by a linkage other than a phosphodiester bond, so long as it does not interfere with hybridization. For example, probes may be peptide nucleic acids (PNAs) in which the constituent bases are joined by peptide bonds rather than phosphodiester linkages. It will be understood that probes may bind target sequences lacking complete complementarity with the probe sequence depending upon the stringency of the hybridization conditions. The probes are preferably directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence (sequence fragment).

Embodiments of the present disclosure also include a method of treating and/or preventing a disease or condition in a subject by administering any of the engineered yeast cells or compositions comprising the engineered yeast cells described herein. In accordance with these embodiments, the methods include administering any of the compositions described herein to the subject. In some embodiments, the composition is administered orally, rectally, parenterally, intramuscularly, intraperitoneally, intravenously, intracerebroventricularly, intracisternally, subcutaneously, via injection or infusion, via inhalation, spray, nasal, vaginal, rectal, sublingual, or topical administration. In some embodiments, the composition is administered orally or rectally.

In some embodiments, the engineered yeast cells are present in the composition at a dose from about 1×10⁵ cells/kg body weight to about 1×10¹⁵ cells/kg body weight of the subject. In some embodiments, the engineered yeast cells are present in the composition at a dose from about 1×10⁵ cells/kg body weight to about 1×10¹⁴ cells/kg body weight of the subject. In some embodiments, the engineered yeast cells are present in the composition at a dose from about 1×10⁵ cells/kg body weight to about 1×10¹³ cells/kg body weight of the subject. In some embodiments, the engineered yeast cells are present in the composition at a dose from about 1×10⁵ cells/kg body weight to about 1×10¹² cells/kg body weight of the subject. In some embodiments, the engineered yeast cells are present in the composition at a dose from about 1×10⁵ cells/kg body weight to about 1×10¹¹ cells/kg body weight of the subject. In some embodiments, the engineered yeast cells are present in the composition at a dose from about 1×10⁵ cells/kg body weight to about 1×10¹⁰ cells/kg body weight of the subject. In some embodiments, the engineered yeast cells are present in the composition at a dose from about 1×10⁵ cells/kg body weight to about 1×10⁶ cells/kg body weight of the subject. In some embodiments, the engineered yeast cells are present in the composition at a dose from about 1×10⁵ cells/kg body weight to about 1×10⁷ cells/kg body weight of the subject. In some embodiments, the engineered yeast cells are present in the composition at a dose from about 1×10⁵ cells/kg body weight to about 1×10⁸ cells/kg body weight of the subject. In some embodiments, the engineered yeast cells are present in the composition at a dose from about 1×10⁵ cells/kg body weight to about 1×10⁹ cells/kg body weight of the subject. In some embodiments, the engineered yeast cells are present in the composition at a dose from about 1×10⁵ cells/kg body weight to about 1×10¹⁰ cells/kg body weight of the subject. In some embodiments, the engineered yeast cells are present in the composition at a dose from about 1×10¹⁰ cells/kg body weight to about 1×10¹⁵ cells/kg body weight of the subject. In some embodiments, the engineered yeast cells are present in the composition at a dose from about 1×10⁸ cells/kg body weight to about 1×10¹² cells/kg body weight of the subject.

In some embodiments, the composition comprising the engineered yeast cells of the present disclosure further comprises at least one pharmaceutically acceptable excipient or carrier. A pharmaceutically acceptable excipient and/or carrier or diagnostically acceptable excipient and/or carrier includes but is not limited to, sterile distilled water, saline, phosphate buffered solutions, amino acid-based buffers, or bicarbonate buffered solutions. An excipient selected and the amount of excipient used will depend upon the mode of administration. An effective amount for a particular subject/patient may vary depending on factors such as the condition being treated, the overall health of the patient, the route and dose of administration, and the severity of side effects. Guidance for methods of treatment and diagnosis is available (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK). For any compositions described herein comprising the engineered yeast cells, a therapeutically effective amount can be initially determined from animal models. A therapeutically effective dose can also be determined from human data which are known to exhibit similar pharmacological activities, such as other adjuvants. The applied dose can be adjusted based on the relative bioavailability and potency of the administered engineered yeast cells and the corresponding proteins or peptides expressed by the engineered yeast cells. Adjusting the dose to achieve maximal efficacy based on the methods described above and other methods as are well-known in the art is well within the capabilities of the ordinarily skilled person in the art.

The compositions described herein may be formulated in a conventional manner using one or more physiologically acceptable carriers comprising excipients and auxiliaries, which facilitate processing of the active ingredients into compositions for pharmaceutical use. Methods of formulating pharmaceutical compositions are known in the art (see, e.g., “Remington's Pharmaceutical Sciences,” Mack Publishing Co., Easton, PA). In some embodiments, the compositions are subjected to tabletting, lyophilizing, direct compression, conventional mixing, dissolving, granulating, levigating, emulsifying, encapsulating, entrapping, or spray drying to form tablets, granulates, nanoparticles, nanocapsules, microcapsules, microtablets, pellets, or powders, which may be enterically coated or uncoated. Appropriate formulation depends on the route of administration. The compositions described herein may be formulated into pharmaceutical compositions in any suitable dosage form (e.g., liquids, capsules, sachet, hard capsules, soft capsules, tablets, enteric coated tablets, suspension powders, granules, or matrix sustained release formations for oral administration) and for any suitable type of administration (e.g., oral, inhalable, topical, injectable, immediate-release, pulsatile-release, delayed-release, or sustained release). The pharmaceutically acceptable compositions may be formulated into pharmaceutical compositions comprising one or more pharmaceutically acceptable carriers, thickeners, diluents, buffers, buffering agents, surface active agents, neutral or cationic lipids, lipid complexes, liposomes, penetration enhancers, carrier compounds, and other pharmaceutically acceptable carriers or agents. For example, the pharmaceutical composition may include, but is not limited to, the addition of calcium bicarbonate, sodium bicarbonate, calcium phosphate, various sugars and types of starch, cellulose derivatives, gelatin, vegetable oils, polyethylene glycols, and surfactants, including, for example, polysorbate 20.

### 3. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

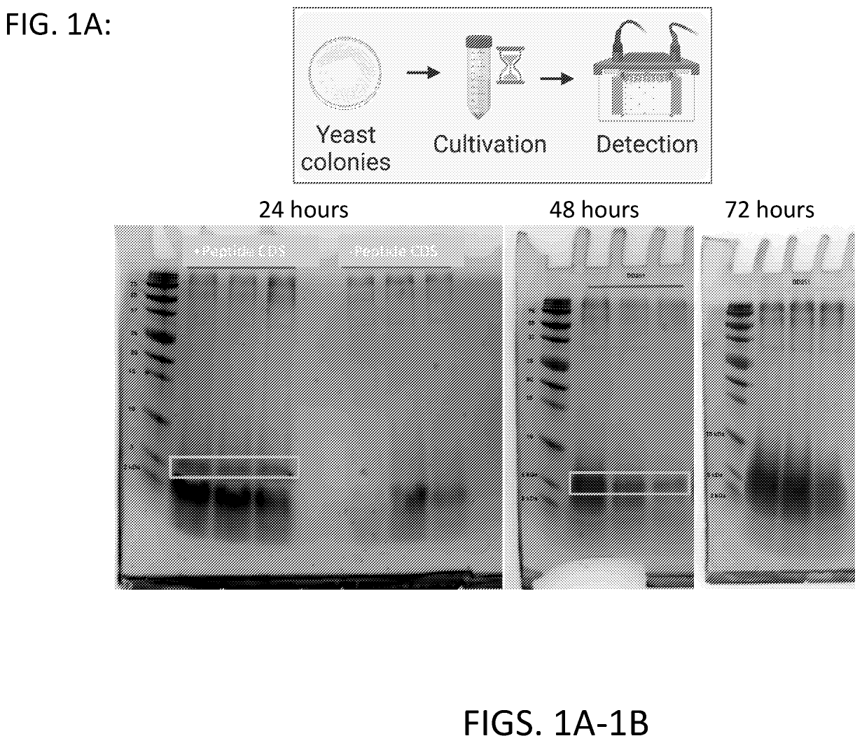

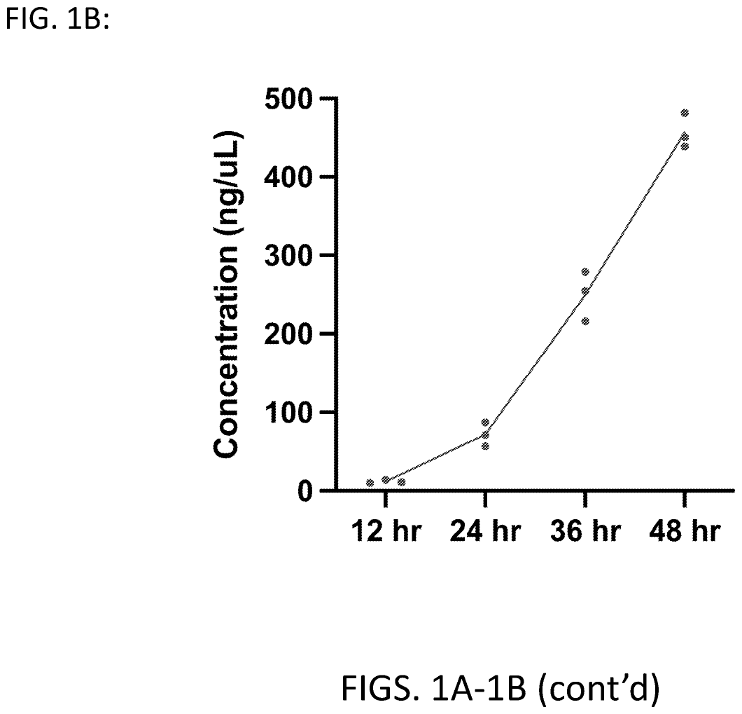

Cloning secretion plasmids. Therapeutic peptide sequence was reverse translated, and codon optimized for expression in S. boulardii. The DNA sequence was cloned into a plasmid backbone containing TDH3 promoter, alpha mating factor signal (αMFprepro), hexahistidine protein tag, c-myc protein tag, TDH1 terminator, URA3 (yeast) marker, 2 μ (yeast) origin, ampicillin (bacteria) marker and ColEl (bacteria) marker via Q5 site directed mutagenesis, yielding DD224 plasmid. DD224 plasmid was then transformed into S. boulardiiΔura3 (DD277) yielding DD314, which was referred to as baseline strain (FIG. 1A).

Detecting secretion in Sb. DD314 was cultured in complete synthetic media (CSM-URA) lacking uracil (CSM-URA) for 24, 36 and 48 hours in bioreactor tubes (50 mL). As negative control, DD316 (DD277 with a non-coding sequence containing plasmid) was cultured. Then culture supernatants were collected by centrifugation and 30 uL of the supernatants were run on Tris-Tricine SDS-PAGE gels. The gels were stained with Coomassie blue and imaged using gel imager (FIG. 1A).

Time course secretion. DD314 was cultured in complete synthetic media (CSM-URA) lacking uracil (CSM-URA) for 12, 24, 36 and 48 hr in Biolector II microfermentation device. At each timepoint, the microfermentation runs were stopped and 20 uL of the supernatant was collected. The supernatant was processed with sandwich ELISA. In this protocol his-tagged therapeutic peptides were selectively bound to the nickel chelates on the bottom of the wells, after wash steps, an anti c-myc tag antibody conjugated with HRP was added to the wells initiating an oxidation reaction with its substrate TMB yielding a chemiluminescence output than was read at OD450 (FIG. 1B).

### Example 2

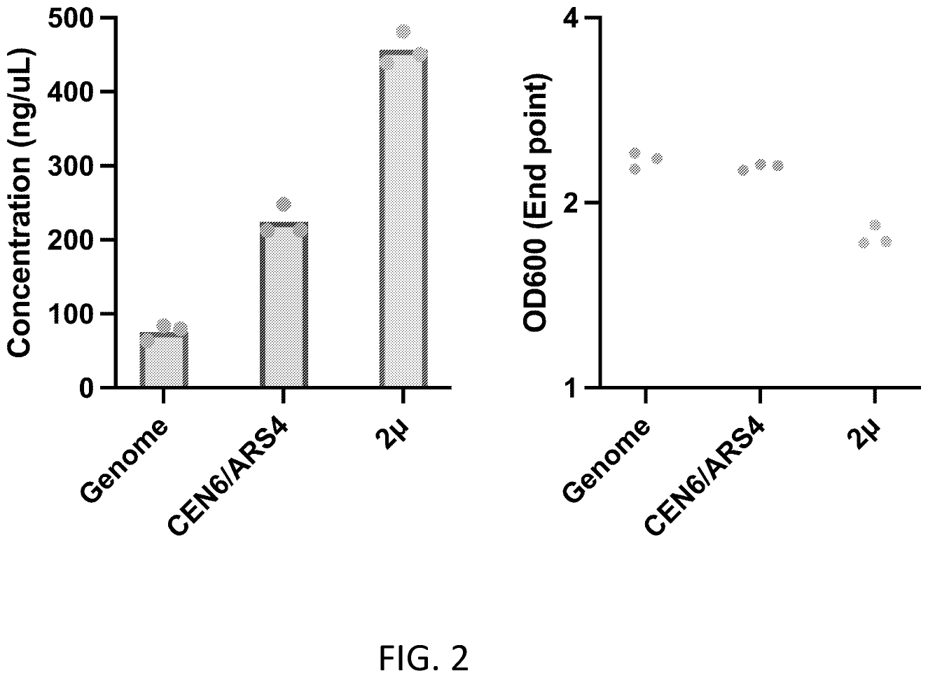

Screening copy-number effect. Additional vectors such as low-copy (CEN6/ARS4) and genomic integration (chromosomal) were constructed. In order to construct low-copy vector, 2 μ origin on DD224 plasmid was swapped with centromeric origin (CEN6/ARS4). In order to construct genomic integration vector, transcriptional unit consisting of TDH3 promoter, alpha mating factor signal (αMFprepro), hexahistidine protein tag, therapeutic peptide sequence, c-myc protein tag, TDH1 terminator was cloned into a repair template plasmid (ISA086) containing 500 base pair long 5′ and 3′ homology arms to the integration site 1 (previously described), yielding DD412 plasmid. DD412 plasmid was then co-transformed into DD277 with Cas9-gRNA co-expression plasmid, ISA1045, enabling Cas9 endonuclease assisted DNA cleavage with homologous recombination assisted editing/repair. This transformation yielded genomic integration of transcriptional unit encoding for therapeutic peptide secretion at the desired integration locus. Once the low-copy and genomic integration S. boulardii strains were constructed, they were cultured in complete synthetic media (CSM-URA) lacking uracil (CSM-URA) for 48 hr in Biolector II microfermentation device. Once the microfermentation finished OD600 values were measured for cell biomass and cell culture supernatants were processed with sandwich ELISA, as described above (FIG. 2).

### Example 3

Screening secretion signals. In addition to alpha mating factor signal (αMFprepro) signal, 5 other secretory leaders were cloned in secretion plasmid. alpha mating factor signal (αMFprepro) were swapped with 2 native leaders; alpha mating factor signal pre- and invertase signal, 3 synthetic/fusion leaders, YAP3-TA57, preOST1-proαMF (I) and preOST1-proαMF (MUT1). These plasmids then transformed to DD277. New S. boulardii strains were cultured in complete synthetic media (CSM-URA) lacking uracil (CSM-URA) for 36 hr in Biolector II microfermentation device. Once the microfermentation finished OD600 values were measured for cell biomass and cell culture supernatants were processed with sandwich ELISA, as described above (FIG. 3).

### Example 4

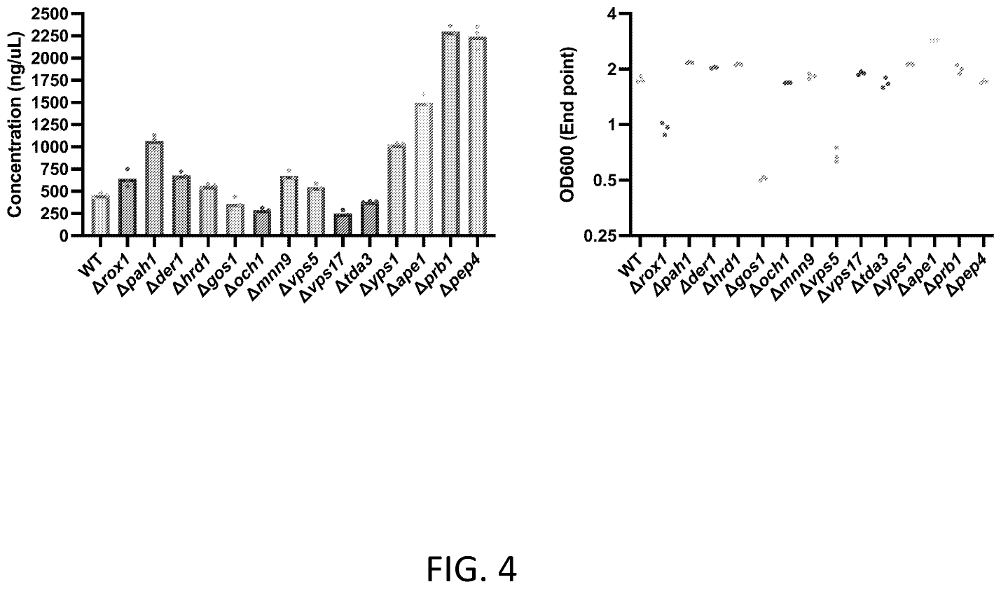

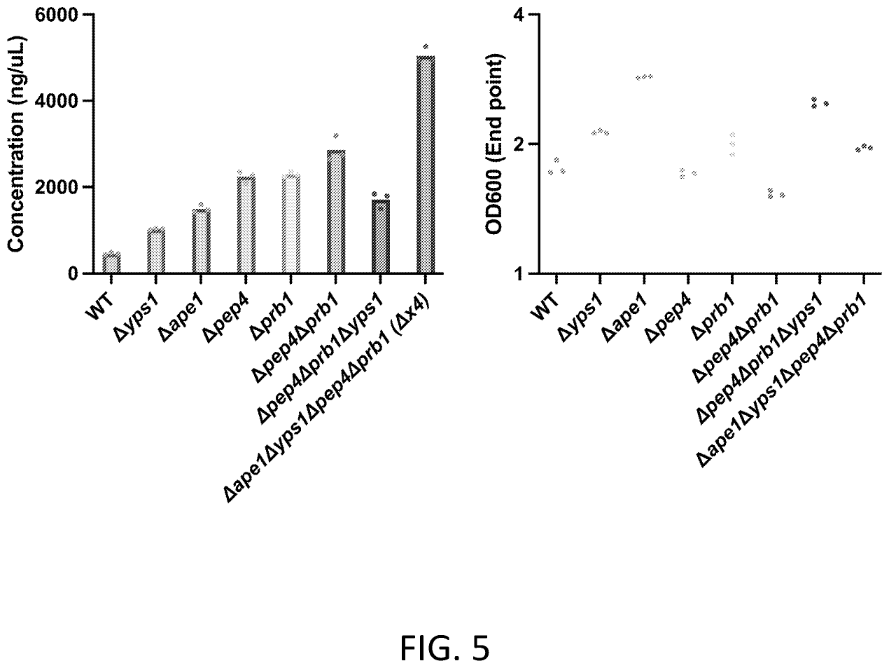

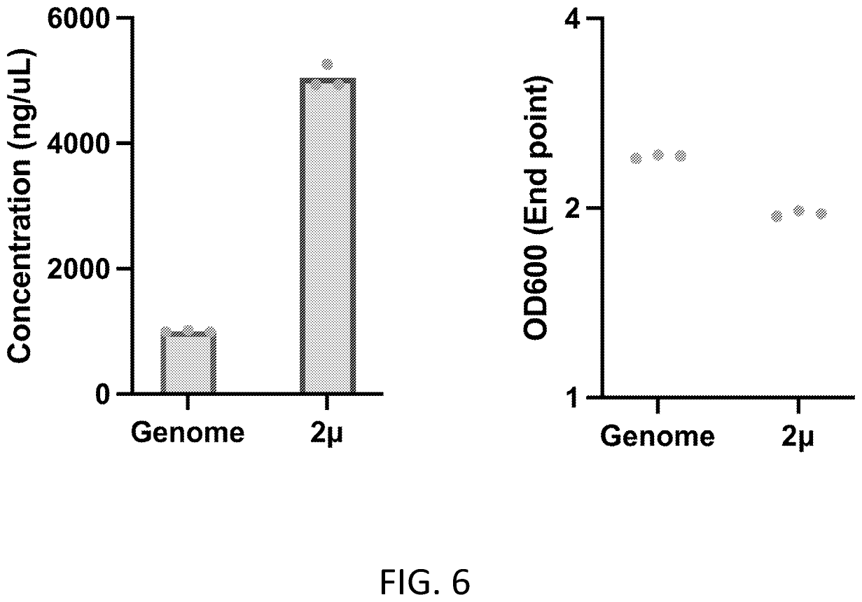

Constructing knockouts in Sb. In order to knock-out genes of interest 20 base-pair long guide RNA sequences were designed to guide Cas9 endonuclease activity. These sequences were ordered as oligonucleotides and cloned into Cas9-gRNA GFP dropout plasmid (SPC1585). Repair templates were ordered as gene fragments and synthesized chemically. Repair templates contained 300-500 bp upstream and downstream sequences of the gene of interest for knocking out, enabling the complete deletion of the gene. The Cas9-gRNA plasmid and amplified repair template was transformed to DD277. Correct edits (deletion of the genes) were confirmed via colony PCR followed by Sanger sequencing. In order to construct double, triple and quadruple knockouts, a consecutive knockout strategy was followed, instead of doing them at the same time. In addition, a genomic integration strain was constructed in quadruple knockout as it was described in “Screening copy-number effect” section (FIGS. 4-6).

Screening knockouts. DD224 plasmid was transformed to knock-out strains. These strains (including the baseline strain DD314) were cultured in complete synthetic media (CSM-URA) lacking uracil (CSM-URA) for 48 hr in Biolector II microfermentation device. Once the microfermentations finished OD600 values were measured for cell biomass and cell culture supernatants were processed with sandwich ELISA, as described above.

Various embodiments of the present disclosure are described herein. Variations of those embodiments may become apparent to those of ordinary skill in the art upon reading the foregoing description. The inventors expect skilled artisans to employ such variations as appropriate, and the inventors intend for the various embodiments of the present disclosure to be practiced otherwise than as specifically described herein. Accordingly, embodiments of the present disclosure include all modifications and equivalents of the subject matter recited in the claims appended hereto as permitted by applicable law. Moreover, any combination of the above-described elements in all possible variations thereof is encompassed by the various embodiments of the present disclosure unless otherwise indicated herein or otherwise clearly contradicted by context.

What is claimed is:

1. An engineered yeast cell comprising:
(i) at least one genetic modification that reduces activity and/or expression of one or more of the following genes: APE1, YPS1, PRB1, PEP4, PAH1, DER1, HRD1, OCH1, MNN9, VPS5, VPS17, TDA3, GOS1, and ROX1; and
(ii) at least one exogenous polynucleotide encoding a target polypeptide or protein, wherein expression of the target polypeptide or protein is increased compared to a yeast cell lacking the at least one genetic modification.

2. The engineered yeast cell of claim 1, wherein the yeast cell is from a probiotic strain.

3. The engineered yeast cell of claim 1, wherein the yeast cell is selected from the group consisting of S. boulardii, S. cerevisiae, P. pastoris, Y. lipolytica, C. albicans, K. marxianus, Debaryomyces hansenii, and Kluyveromyces lactis.

4. The engineered yeast cell of claim 3, wherein the yeast cell is from an S. boulardiiΔura3 strain (DD277).

5. The engineered yeast cell of any of claims 1 to 4, wherein the at least one genetic modification is a genetic knockout.

6. The engineered yeast cell of any of claims 1 to 5, wherein the at least one genetic modification comprises a genetic knockout of YPS1.

7. The engineered yeast cell of any of claims 1 to 5, wherein the at least one genetic modification comprises a genetic knockout of PRB1.

8. The engineered yeast cell of any of claims 1 to 5, wherein the at least one genetic modification comprises a genetic knockout of PEP4.

9. The engineered yeast cell of any of claims 1 to 5, wherein the at least one genetic modification comprises a genetic knockout of APE1.

10. The engineered yeast cell of any of claims 1 to 5, wherein the at least one genetic modification comprises a genetic knockout of PRB1 and PEP4.

11. The engineered yeast cell of any of claims 1 to 5, wherein the at least one genetic modification comprises a genetic knockout of YPS1, PRB1, PEP4, and APE1.

12. The engineered yeast cell of any of claims 1 to 11, wherein the exogenous polynucleotide comprises a promoter upstream of the target polypeptide or protein.

13. The engineered yeast cell of any of claims 1 to 11, wherein the exogenous polynucleotide comprises a secretion signal upstream of the target polypeptide or protein.

14. The engineered yeast cell of claim 13, wherein the secretion signal comprises an alpha mating factor secretion signal, an invertase secretion signal, a YAP3-TA57 secretion signal, a preOST1-proαMF (I) secretion signal, or a preOST1-proαMF (MUT1) secretion signal, and any combinations thereof.

15. The engineered yeast cell of any of claims 1 to 14, wherein the target polypeptide or protein is expressed and secreted from the engineered yeast cell.

16. The engineered yeast cell of any of claims 1 to 14, wherein the target polypeptide or protein is expressed on the surface of the engineered yeast cell.

17. The engineered yeast cell of any of claims 1 to 16, wherein expression or secretion of the target polypeptide or protein is increased at least 1.2-fold compared to a yeast cell lacking the at least one genetic modification.

18. The engineered yeast cell of any of claims 1 to 17, wherein the target polypeptide or protein is at least one of an anti-cancer agent, an immune regulator, an anti-pathogenic agent, and/or a metabolic regulator.

19. The engineered yeast cell of any of claims 1 to 18, wherein the target polypeptide or protein is at least one of a DARPin, a lectin, a monoclonal antibody, a F (ab′) 2 fragment, a Fab′/Fab fragment, a diabody, a scFv, a nanobody, and/or an affibody. 20 A composition comprising any of the engineered yeast cells of any one of claims 1 to 19.

21. The composition of claim 20, wherein the composition is lyophilized.

22. The composition of claim 20, wherein the composition is in wet form.

23. The composition of claim 20, wherein the composition is in frozen form.

24. The composition of any one of claims 20 to 23, wherein the composition is formulated as a food product.

25. A method of treating and/or preventing a disease or condition in a subject, the method comprising administering the composition of any one of claims 19 to 24 to the subject.

26. The method of claim 25, wherein the engineered yeast cells are present in the composition at a dose from about 1×10⁵ cells/kg body weight to about 1×10¹⁵ cells/kg body weight of the subject.

27. The method of claim 25 or claim 26, wherein the composition is administered orally or rectally.

28. A method of enhancing expression and/or secretion of a target polypeptide or protein in an engineered yeast cell, the method comprising:
making at least one genetic modification to the engineered yeast cell that reduces activity and/or expression of one or more of the following genes: APE1, YPS1, PRB1, PEP4, PAH1, DER1, HRD1, OCH1, MNN9, VPS5, VPS17, TDA3, GOS1, and ROX1;
wherein expression of the target polypeptide or protein is increased compared to a yeast cell lacking the at least one genetic modification.
